(19) United States
(12) Patent Application Publication
(10) Pub. No.: US 2025/0295954 A1
(43) Pub. Date: Sep. 25, 2025

(54) SYSTEM AND METHOD OF AUTO-CLASSIFICATION OF IMPACTS

(71) Applicant: Sports & Wellbeing Analytics Limited, Swansea (GB)

(72) Inventors: Chris Jones, Swansea (GB); Rowan Brown, Swansea (GB)

(21) Appl. No.: 19/085,235

(22) Filed: Mar. 20, 2025

(30) Foreign Application Priority Data
Mar. 21, 2024 (GB) 2404075.0

(51) Int. Cl. A63B 24/00; A63B 71/08; G06N 20/00

(52) U.S. Cl. CPC A63B 24/0062; A63B 71/085; G06N 20/00; A63B 2220/53; A63B 2220/836

(57) ABSTRACT

A computer implemented method of generating a feature array configured for training a classifier of impact data measured by a mouthguard includes receiving signal data, receiving impact classification data representative of feature information that specifies the class of impact of each received signal data and storing the impact classification in a response array, extracting one or more features from the signal data and storing in an array, comparing each extracted feature to the corresponding response array element to select the set of features that respectively satisfy a classification relevance threshold indicative of a classification relevance, and, responsive to the number of selected features being less than a feature threshold, iteratively rotating the direction of respective x, y, z components of the rotational velocity time series data relative to corresponding x, y, z components of the linear acceleration time series data.

### CROSS REFERENCE TO RELATED APPLICATION

This application claims the benefit of United Kingdom Patent Application No. GB2404075.0, filed on Mar. 21, 2024. The prior application is incorporated herein by reference in its entirety.

### FIELD

The present invention relates to a method and system for generation of a training dataset feature array, training of a classifier, and classification of acceleration data.

### BACKGROUND

Participants in sports, particularly contact sports, such as, for example, Rugby Football, American (NFL) Football, Boxing, Mixed Martial Arts (MMA), Association Football (soccer) etc., and particularly professional participants in sports, receive impacts from collisions with other participants during match play. These impacts may be, and often are, heavy, violent impacts and may include direct head impacts. A head of the participant receiving such a heavy impact may be moved violently and be subject to great accelerative forces, termed hereinafter “head impact event.” While an individual head impact event may not cause a concussion event of its own effect, the effect of such violent movements can be cumulative. Therefore, when a participant has sustained a critical number of violent head movements over a certain period of time it is advisable to prevent the participant from playing in further games to reduce the risk of the participant being exposed to the chance of a potential serious head injury event, for example, one that might cause concussion, or further concussion, or other serious injury. It is possible to monitor impacts sustained by participants, not only during individual matches, but also over the course of their careers so that a medical team or coach, for example, can be made aware of the situation. A participant may be advised as to when they should next play or train.

Historically, it has been difficult to monitor head impact events, particularly the linear and rotational acceleration and forces actually encountered by a participant during a game. One method used to date in professional games has involved a “spotter” for each participant. The “spotter” typically may be located in the audience at the sporting event, or may be viewing the sporting event remotely via a video link. Each “spotter” is tasked with monitoring impacts, or blows, received by their assigned participant. If the “spotter” observes an impact event that they deem to be a head impact event, they can coordinate the stoppage of play in order to remove their assigned participant from the field-of-play to be assessed by a qualified medical practitioner. However, such assessment is subjective and does not provide an accurate indication of the forces sustained, which may be different for each participant involved in a collision leading to one or more head impact events. Furthermore, it is highly expensive, because personnel may be required for each participant on the field-of-play. Additionally, such a monitoring environment is generally not available in amateur-level contact sports matches and thus amateur participants are not afforded the same level of monitoring for head impact events as their professional counterparts.

In an attempt to objectively measure the accelerations or forces sustained by a participant, it has been suggested to use monitoring units, to be worn by the participant. Such monitoring units include sensors, for example inertial measurement units, which operate to monitor acceleration experienced by a wearer during the course of match play. Data produced by the sensors of the monitoring units can be stored in an on-board memory and/or transmitted to a monitoring station for review by a technician.

One such know monitoring unit comprises a removable intra oral appliance such as a mouthguard or gumshield (hereinafter “mouthguard”) embedded with sensors. Sometimes referred to herein as an instrumented mouthguard or (IMG). Such types of units have been increasingly adopted, because they are worn within a mouth of the participant (and so their use is unlikely to be prohibited by the regulation of many sports, because they are not worn outside the body). Furthermore, they are seen as potentially more accurate than external units worn on the body, or located in helmets, because they may measure more accurately the sustained acceleration by a participant due to an impact on the participant (from which acceleration an impact force can be derived). This is because they are typically worn against teeth of the upper jaw, and since the upper jaw is a fixed part of the head (as opposed to the lower jaw, which is moveable relative to the rest of the head), will move with the head. Therefore, the unit will typically undergo the same movement during an impact event (and experience the same forces and accelerations) as that experienced by the head itself. Such units are seen to be beneficial, because no adhesions of sensors to the head or neck of the participant are required.

The data from such IMGs may be transmitted to an external monitoring unit, either live while being used, or after a match has finished. The movements of the IMG, and therefore the movement of the head of the person wearing the IMG, may be analysed to assess and characterise the types of impact events experienced. When IMGs are customised for a specific wearer there are many factors that can be different between respective customised IMGs and therefore can contribute to variations in the quality of the data recorded and transmitted by respective customised IMGs. This can result in the not only variations in the characterisation of the impacts, but also to inconsistencies between the response of sensors mounted within the IMG.

Aspects and embodiments in accordance with the present invention have been devised with the foregoing in mind.

### SUMMARY

According to a first aspect of the invention there is provided a computer implemented method of generating a feature array configured for training a classifier of impact data measured by a mouthguard, the method comprising:
a) receiving signal data, the received signal data representative of linear acceleration and rotational velocity measurement time series signals indicative of an impact measured from an instrumented mouthguard;
b) receiving impact classification data representative of feature information that specifies the class of impact of each received signal data and storing the impact classification in a response array;
c) extracting one or more features from the signal data and storing in an array, the one or more features corresponding to a single impact;
d) comparing each extracted feature to the corresponding response array element to select the set of features that respectively satisfy a classification relevance threshold indicative of a classification relevance;
e) responsive to the number of selected features being less than a feature threshold, iteratively rotating the direction of respective x, y, z components of the rotational velocity time series data relative to corresponding x, y, z components of the linear acceleration time series data and performing steps c) to d) until the number of selected features meet the feature threshold.

Rotating the direction of respective x, y, z components of the rotational velocity time series data relative to corresponding x, y, z components of the linear acceleration time series data provides a customisable method of generating a training dataset to account for variability in the data received. For example, the linear acceleration data and rotational velocity data may be measured by two separate components mounted in an IMG and thus could have different relative orientations. Due to this, the respective corresponding orthogonal axes of the data received could be misaligned and the relative positions unknown. While there are methods to “calibrate” the relative positions, to do so for each IMG manufactured would increase time for action and costs. Such a method of producing a training dataset removes the need for the absolute position of the two senses in the IMG to be known or calibrated. This simplifies the manufacture of such IMGs reduces the overall cost thereof.

Optionally, the one or more extracted features are time domain features and/or frequency domain features.

Optionally, a further pre-processing step of rotationally aligning respective x, y, z components of the rotational velocity time series data to match that of corresponding x, y, z components of the linear acceleration time series data is performed prior to step c) of the first embodiment of the present invention. Such a step aligns the axes of the gyroscope to the accelerometer, which may be separate devices located in an IMG. Therefore, by providing an alignment technique allows the data taken from the two separate devices to be calibrated so as to be on the same axis. This provides an element of flexibility in the manufacturing process of the IMGs because the location of sensors may vary from model to model. The alignment step described above allows the method to be implemented on any IMG, or other device with a gyroscope and accelerometer mounted therein, independent of the location of the sensors.

Optionally, the received data is filtered to remove cyclically occurring noise prior to step c) of the first embodiment of the present invention. Removal of such moderate to high frequency noise prepares the received signal to be subsequently processed.

Optionally, the received signal data comprises one or more discrete signal data packets, and responsive to one or more of the discrete signal data packets comprising no linear acceleration and rotational velocity measurement time series signal data indicative of an impact measured from an instrumented mouthguard, reconstructing the signal data. Missing data from the received signal can result in extraction of features that are not true to the measurement made and thus reconstructing improves the features extracted. Accordingly, the training set generated from reconstructed data is improved.

Optionally, the signal data is reconstructed by interpolation.

Optionally, the classification relevance threshold is calculated by a minimum redundancy maximum relevance (MRMR) algorithm, which provides a “rank” of all features generated. Only the features which most closely represent the correct response or label for that particular impact that is represented by the signal is chosen for use in the training set. Therefore, an improved training dataset is realised.

Optionally, the time domain features and/or frequency domain features are any one or more of the following: Minimum, Maximum, Mean, Standard Deviation, Root-Mean-Square value, Mean Absolute Deviation, Skewness, Kurtosis, Autocorrelation, cross-correlation, Spectral Power, peak magnitude, and peak position.

According to a second aspect of the present invention there is provided a computer implemented method of training a machine learning model for classification of impact data measured by a mouthguard comprising:
a) providing the feature dataset and the response array according to the first aspect of the present invention;
b) partitioning the feature dataset into a feature array training data dataset and a feature array test dataset;
c) partitioning the response array into a response array training dataset and a response array test dataset, the elements of the response array training dataset corresponding to the elements of the feature array training dataset and the elements of the response array test dataset corresponding to the feature array test dataset;
d) inserting the feature array training dataset into one or more classifier models and training the selected classifier model using the training dataset;
e) evaluating each classifier model accuracy using the test dataset to determine a value indicative of the correct classifications on the response array training dataset;
f) comparing the classifier model accuracy to a predetermined accuracy;
g) iteratively updating the hyperparameters of the selected classifier model and performing steps c) to d) until the classifier model accuracy is over the predetermined accuracy.

A classifier trained with the training dataset generated by the first aspect of the present invention will result in a better trained classifier for classification of impacts. Particularly, where two sensors are used in an IMG resulting in the axes of the data between the two being indeterminate. Such a method makes the training agnostic to the placement and relative disposition of the sensors mounted in the IMG.

Optionally, responsive to the model accuracy determined in step g) of the second aspect of the present invention failing to exceed the predetermined threshold:
h) iteratively rotating one or more of the axes of the rotational velocity time series data relative to the corresponding linear acceleration axes and performing steps c) to g) until the model accuracy determined in step g) attains or exceeds the predetermined accuracy.

If the trained classifier does not yield the desired accuracy, this provides another option to improve the classification.

Optionally, the predetermined accuracy is the area under the curve and/or receiver operating characteristic.

According to a third aspect of the present invention there is provided a computer implemented method of classifying impact data measured by an instrumented mouthguard comprising the steps of:
a) receiving signal data, the received signal data representative of linear acceleration and rotational velocity measurement time series signals indicative of an impact measured from an instrumented mouthguard;
b) providing the trained machine learning model of the second aspect of the present invention; and
c) inserting the received signal data into the trained machine learning model.

A method classification that uses the trained classifier of the second aspect of the invention such as the one above results in a more accurate classification of impact events from data received from IMGs as described above.

According to a fourth aspect of the present invention there is provided a computer readable storage medium containing instructions thereon implementable by a computer to carry out any of the first, second, or third aspects of the present invention.

According to a fifth aspect of the present invention, there is provided a data processing apparatus, comprising data processing resources configured to implement the method of the first aspect for generating a feature array configured for training a classifier of impact data measured by a mouthguard.

According to a sixth aspect of the present invention there is provided a data processing apparatus, comprising data processing resources configured to implement the method of the second aspect for training a machine learning model for classification of impact data measured by a mouthguard.

According to a seventh aspect of the present invention there is provided a data processing apparatus, comprising data processing resources configured to implement the method of the third aspect for classifying impact data measured by an instrumented mouthguard.

According to an eighth aspect of the present invention there is provided a classifier comprising data processing apparatus according to the seventh aspect.

### BRIEF DESCRIPTION OF THE DRAWINGS

One or more specific embodiments in accordance with aspects of the present invention will be described, by way of example only, and with reference to the following drawings in which:

FIG. 1 illustrates a mouthguard according to one or more embodiments of the present invention in which embedded and/or encapsulated components are arranged in a first arrangement;

FIG. 2 illustrates a mouthguard according to one or more embodiments of the present invention in which embedded and/or encapsulated components are arranged in a second arrangement;

FIG. 3 illustrates a system for providing a monitoring environment for monitoring acceleration sustained by participants in a sporting event;

FIG. 4 illustrates a schematic block diagram of the components of a mouthguard according to one or more embodiments of the present invention;

FIG. 5 is an illustrative example of linear acceleration data as a component of time measured by a mouthguard according to one or more embodiments of the present invention;

FIG. 6 is an illustrative example of the linear acceleration data measured by a mouthguard in the spectral domain according to one or more embodiments of the present invention;

FIG. 7 is an illustrative process flow control diagram of a computer implemented method of training a classifier according to one or more embodiments of the present invention;

FIG. 8 is an illustrative process flow control diagram of a data process flow according to one or more embodiments of the present invention;

FIG. 9a is an illustrative process flow control diagram of a method for generating a training dataset;

FIG. 9b is an example of features extracted from data recorded by a mouthguard according to one or more embodiments of the present invention;

FIG. 9c is an example of features extracted from data recorded by a mouthguard according to one or more embodiments of the present invention;

FIG. 10. is an example histogram of predictor importance rank as a function of feature and a display of a ranked feature plotted against another ranked feature for a first sensor orientation according to one or more embodiments of the present invention;

FIG. 11. is an example histogram of predictor importance rank as a function of feature and a display of a ranked feature plotted against another ranked feature for a second sensor orientation according to one or more embodiments of the present invention;

FIG. 12 is an illustrative process control flow diagram of training a classifier according to one or more embodiments of the present invention;

FIG. 13 is an illustrative confusion matrix output from a trained classifier according to one or more embodiments of the present invention;

FIG. 14 is an example receiver operator characteristic curve according to one or more embodiments of the present invention;

FIG. 15 is an illustrative confusion matrix output from a trained classifier according to one or more embodiments of the present invention;

FIG. 16 is an example receiver operator characteristic curve according to one or more embodiments of the present invention;

FIG. 17 is an illustrative confusion matrix output from a trained classifier according to one or more embodiments of the present invention;

FIG. 18 is an example receiver operator characteristic curve according to one or more embodiments of the present invention; and

FIG. 19 is an example process control flow diagram of training a classifier according to one or more embodiments of the present invention.

### DETAILED DESCRIPTION

An IMG (also known as a gumshield or mouthguard) is a piece of protective equipment worn by participants in sports, particularly contact sports. An IMG is typically worn in an upper part of the mouth of the participant and is generally configured to cover at least a portion of the upper teeth of the participant. Most typically, a mouthguard is configured to cover at least a portion of a vestibular (outer) surface of upper teeth of the wearer, at least a portion of a palatal (inner) surface of upper teeth of the wearer, and at least a portion of incisal and occlusal surfaces (i.e. “biting” and “chewing” surfaces) of upper teeth of the wearer.

In general outline, an IMG according to one or more embodiments of the present invention can form a part of a system for the detection, measurement, characterisation, transmission, and/or reporting of impact events causing acceleration to be experienced by participants. Sensor components and/or monitoring element components located in the IMG are used to monitor accelerations experienced by participants and data representative of such accelerations can be conveyed to a monitoring station for review by a technician, for example, a trained medical professional. This can allow the technician to make a decision regarding whether or not a participant in a sports match is fit to continue playing (e.g. following a particularly heavy head impact event) or should be removed from play and referred for further testing with a medical professional.

In the present description, the phrase “head impact event” relates to both direct impacts to the head and indirect impacts. That is, where the head receives a blow directly, or when a blow is sustained to some other body part and the force of the blow causes, amongst other things, an acceleration of the head. Further, reference is made to a participant sustaining an impact and their head experiencing an acceleration because of the impact. The acceleration of the head may be as a result of an impact directly to the head (i.e. a force is exerted on the head directly), or as result of an impact to another part of the body, but the result of which is that force is transmitted to the head from the point-of-impact through the body and neck. Such an acceleration may be termed an impact acceleration.

The sensor and/or monitoring element components are embedded and/or encapsulated in material from which the IMG is formed.

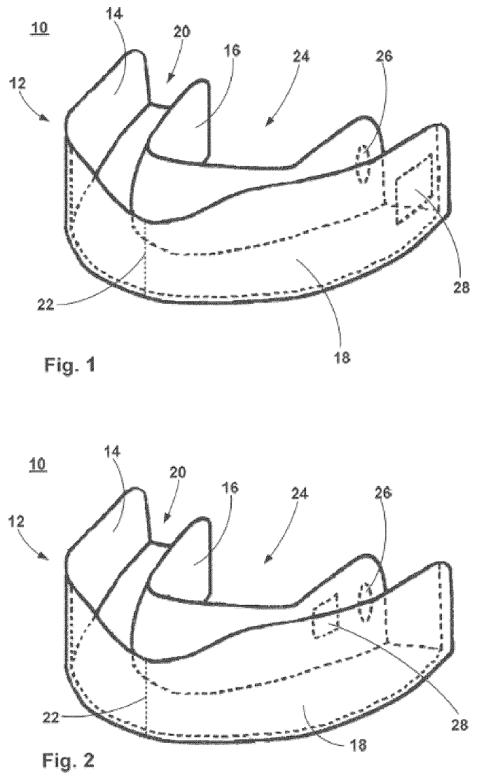

FIG. 1 illustrates an IMG 10 according to one or more embodiments of the present invention in which embedded and/or encapsulated components are arranged in a first arrangement. FIG. 2 illustrates an IMG 10 according to one or more embodiments of the present invention in which embedded and/or encapsulated components are arranged in a second arrangement.

In the illustrated IMG 10 of FIGS. 1 and 2, components are shown positioned in walls of the IMG that are locatable at the rear of a mouth of a wearer when the IMG is located correctly in the mouth.

The components are connected electronically by means of wires or circuit board (which may be flexible) and are communicatively coupled to a transceiver for transmitting data received from the components to a monitoring station in real-time. These components operate to collect and process impact event data, which can then be transmitted to the monitoring station via the transceiver.

Various terms used in dentistry are used in describing the IMG 10 of one or more embodiments of the present invention. The terms used in this disclosure are listed below:
Anterior—The direction towards the front of the head or the lips, as opposed to posterior, which refers to the directions towards the back of an individual's head. The term anterior teeth refers to incisors and canines, as opposed to premolars and molars, which are posterior teeth;
Distal—The direction towards the gums beyond the tooth furthest from the midline (i.e. the ‘most posterior tooth’ or last tooth) in each quadrant of a dental arch, as opposed to mesial, which refers to the direction towards the midline;
Incisal—The direction towards the biting edge of front teeth. This is a related term to occlusal, which relates to the analogous location on rear teeth;
Mandibular—Relating to the mandible, or lower jaw;
Maxillary—Relating to the maxilla, or upper jaw;
Mesial—The direction towards the midline in a dental arch, as opposed to distal, which refers to the direction towards the gums beyond the tooth furthest from the anterior midline (the ‘most posterior tooth’ or last tooth) in each quadrant;
Midline—Roughly, an imaginary vertical line dividing the left and right sides of the mouth at the teeth;
Occlusal—The direction towards the biting surface of rear teeth. This is a related term to incisal, which relates to the analogous location on anterior teeth;
Palatal—The side of a tooth adjacent to (or the direction towards) the palate, as opposed to vestibular, which refers to the side of a tooth adjacent to (or the direction towards) the inside of the cheek or lips of the mouth respectively;
Posterior—The direction towards the back of an individual's head, as opposed to anterior, which refers to the directions towards an individual's lips. The term posterior teeth refers to premolars and molars, as opposed to incisors and canines, which are anterior teeth;
Quadrant—The arrangement of teeth in a mouth is divided into four quarters. Upper and lower sets of teeth form an oval, which is divided into quadrants:
   Upper right quadrant: upper right first incisor to upper right wisdom tooth;
   Upper left quadrant: upper left first incisor to upper left wisdom tooth;
   Lower right quadrant: lower right first incisor to lower right wisdom tooth;
   Lower left quadrant: lower left first incisor to lower left wisdom tooth; and
Vestibular—The side of a tooth that is adjacent to (or the direction towards) the inside of the cheeks and lips, as opposed palatal, which refers to the side of a tooth adjacent to the palate.

Additionally, reference is made to monitoring acceleration. In at least some implementations, a device used to measure acceleration is termed an “accelerometer”. The terms “acceleration measurement”, “acceleration monitoring” and the like include use of devices known as “accelerometers”. The terms may be used interchangeably depending on context.

### Example IMG

As illustrated in FIGS. 1 and 2, the IMG 10 comprises a body 12 that defines a formation to be located around at least a portion of maxillary teeth of a wearer (i.e. teeth in the upper jaw of the wearer—hereinafter “upper teeth”), to cover, surround, and/or envelope the upper teeth of the wearer.

The body 12, is formed from a plastics, resin, and/or rubber material. The body 12 comprises a first wall 14 configured to cover at least a portion of an outer surface of the upper teeth of the wearer (i.e. the surface of the upper teeth that faces the inside of the upper lip and the cheek). In dentistry terminology this surface is known as a vestibular surface.

The body 12 comprises a second wall 16 configured to cover at least a portion of an inner surface of the upper teeth of the wearer (i.e. the surface of the upper teeth that faces the palate). In dentistry terminology this surface is known as a palatal surface.

The body 12 comprises a third wall 18 connecting the first and second walls 14, 16 and configured to cover at least a portion of biting edges and chewing surfaces of the upper teeth of the wearer (i.e. the edges and surfaces of the upper teeth that are opposed to the lower teeth). In dentistry terminology, these surfaces are known as incisal and occlusal surfaces.

The first wall 14, the second wall 16 and the third wall 18 of the body 12 define a channel 20 for receiving a plurality of teeth of a wearer. In the illustrated examples of FIGS. 1 and 2, the channel 20 is structured such that, when worn, it covers teeth that include the incisors of a wearer when the IMG 10 is inserted.

In plan view, the body 12 of the IMG 10 presents a generally symmetrical U-shaped configuration with “arms” extending away from a mid-line (denoted by a dashed line 22 in FIGS. 1 and 2). The first wall 14, the second wall 16, and the third wall 18 in one arm define a portion of the channel 20 that can receive teeth of an upper left quadrant. The first wall 14, the second wall 16, and the third wall 18 in the other arm define a portion of the channel 20 that can receive teeth of an upper right quadrant.

The IMG 10 also defines an open area 24, located between the two arms, which can allow a tongue of the wearer to touch their upper palate when the IMG 10 is being worn. This may allow the user to maintain verbal communication with other participants (e.g. teammates) without requiring removal of the IMG.

The IMG 10 includes a power source 26 (e.g. an electrical power battery) that is electrically connected to a system for monitoring acceleration 28. Typically, the power source 26 is of a type compatible with a wireless charger to allow recharging of the power source, i.e. the power source 26 may be wirelessly rechargeable, which allows the power source 26 to be charged/recharged without requiring removal from the IMG 10.

In the illustrated example of FIG. 1, the power source 26 and the system for monitoring acceleration 28 are located in a portion of the same arm of the IMG. The portion in which they are located is in a distal direction from the midline 22. The power source 26 is located in the second wall 16 and the system for monitoring acceleration 28 is located in the first wall 14. The power source 26 and system for monitoring acceleration 28 are electrically connected using a suitable connection (not shown) that runs from the power source 26, through the third wall 18 to system for monitoring acceleration 28.

Optionally, the power source 26 and/or the system for monitoring acceleration 28 may be located in a different area of the IMG 10 in one or more embodiments. FIG. 2 illustrates another example, in which the power source 26 and the system for monitoring acceleration 28 are, again, located in a portion of the same arm of the IMG. The portion in which they are located is in a distal direction from the mid-line 22. However, in the example illustrated in FIG. 2, both the power source 26 and the system for monitoring acceleration 28 are located in the second wall 18.

In the illustrated examples of FIGS. 1 and 2, the components of the IMG 10, described above, are encapsulated (i.e. wholly embedded) within material forming the mouth-guard 10.

### System Diagram

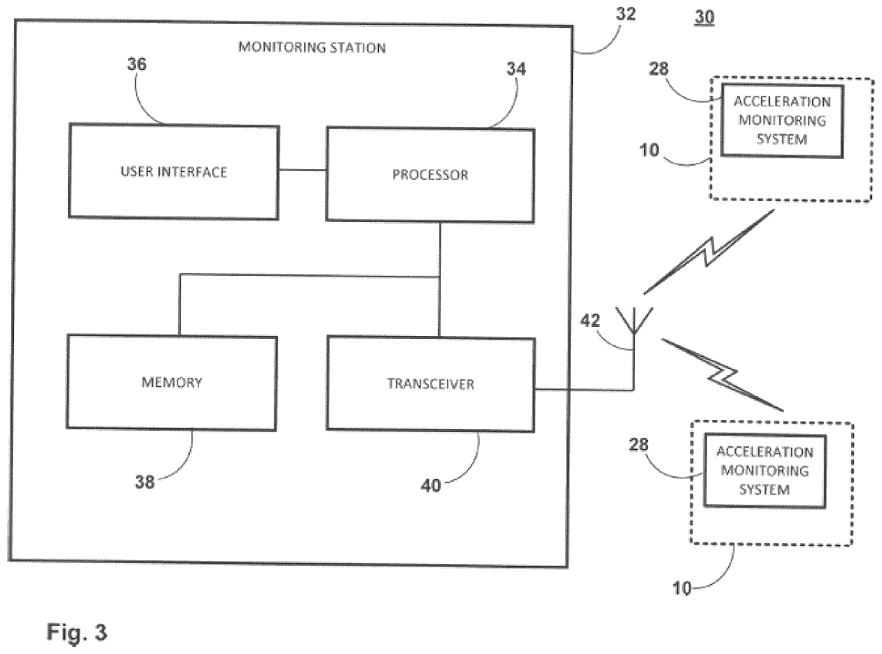

FIG. 3 illustrates a system 30 for providing a monitoring environment for monitoring acceleration and motion as a function of time sustained by participants in a sporting event.

The system 30 operates to aggregate data representative of temporal acceleration that occurs during impact events, the data being received from the system for monitoring acceleration 28 in IMGs 10 worn by game participants. The data can be conveyed to technicians, via the system, for assessing the seriousness of one or more impact events or merely recording an event to be used in analysis on aggregated impact data.

The system 30 comprises a monitoring station 32 that is in wireless communication with one or more systems for monitoring acceleration 28. The monitoring station 32 can communicate data received from the one or more systems for monitoring acceleration 28 to one or more devices (not shown in FIG. 3) either wirelessly or by wired communication link.

The monitoring station 32 includes a processor 34, a user interface 36, memory 38, and a transceiver 40. The monitoring station 32 wirelessly receives data representative of accelerations experienced by participants from each of the systems for monitoring acceleration 28. Signals from each of the systems for monitoring acceleration 28 are received at an antenna 42 coupled to the transceiver 40. The received signals are passed to the processor 34, which operates to process the data. The processed data is communicated to a memory 38 for storage and can also be communicated to the user interface 36, which is configured for communicating the data to a display device (e.g. via a communications network).

### IMG Component System

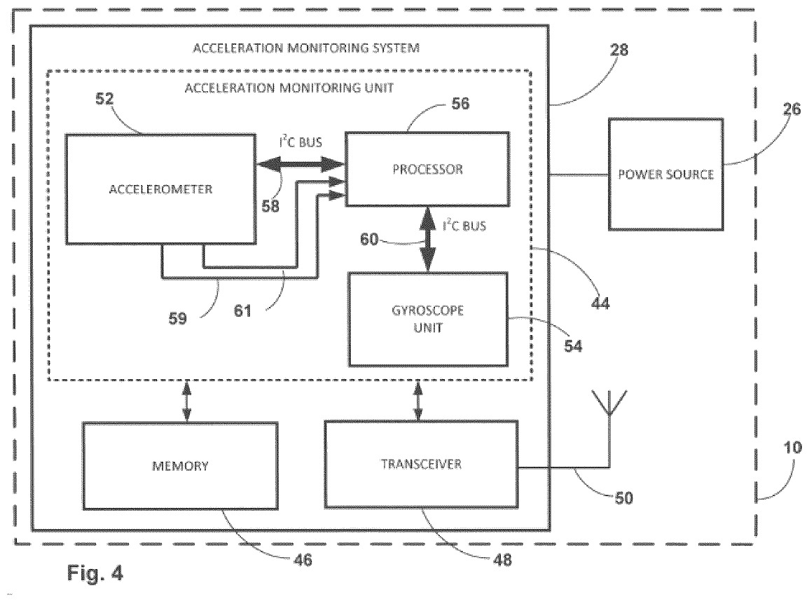

FIG. 4 illustrates components of the IMG 10 (e.g. the power source 26 and the system for monitoring acceleration 28) in more detail such components collectively, individually or in sum or in part providing processing resources for the IMG 10.

The system for monitoring acceleration 28 comprises an acceleration monitoring unit 44, a memory 46 and a transceiver 48. The acceleration monitoring unit 44 is operative to monitor and measure acceleration experienced by a wearer of the IMG 10. The acceleration monitoring unit 44 is electrically coupled to the memory 46, which serves to store data representative of acceleration monitored and measured by the acceleration monitoring unit 44. The acceleration monitoring unit 44 is also electrically coupled to the transceiver 48, which is operative to communicate a data signal containing data representative of acceleration monitored and measured by the system for monitoring acceleration to the monitoring station 32 via antenna 50. External data can also be received by the system for monitoring acceleration 28 from an external source via the antenna 50 and transceiver 48. Received external data may comprise, for example, a negative-acknowledgement signal (e.g. to indicate an error in data previously sent from the system for monitoring acceleration 28 and to request that the data be re-sent), software updates, etc.

The acceleration monitoring unit 44 comprises a three-axis linear accelerometer 52, a gyroscope unit 54 and a processor 56. Each of the acceleration monitoring unit 44 and the gyroscope unit 54 are communicatively coupled to the processor 56 by way of Inter-Integrated Circuit (I²C) buses 58, 60 respectively.

The accelerometer 52 is operative to monitor linear accelerations of the mouth-guard 10. The accelerometer 52 is operative to measure a linear acceleration in each orthogonal direction (x, y, z), e.g. of a Cartesian coordinate reference frame. A combination of respective acceleration values may be used to derive a linear acceleration vector.

The gyroscope unit 54 is operative to measure angular velocity to provide data representative of angular rotation. The gyroscope unit 54 is operative to measure angular velocity with respect to each orthogonal direction (x, y, z), e.g. of a Cartesian coordinate reference frame. A combination of respective angular velocity values may be used to derive a angular velocity vector.

The accelerometer 52 and the gyroscope unit 54 are operative to monitor attributes of the environment of the IMG 10 over time to determine a linear acceleration of the system for monitoring acceleration 28 and an angular velocity of the system for monitoring acceleration 28. Using data indicative of linear acceleration and angular velocity, which is communicated to the processor 56, the processor 56 is able to determine the fact of an event causing acceleration of a particular magnitude and a rotation. This data can be used in the system for monitoring acceleration 28 and/or the monitoring station 32, to calculate a vector representative of a magnitude of the linear acceleration and a vector representative of an angular velocity experienced by the system for monitoring acceleration 28.

The system for monitoring acceleration 28 of the IMG 10 provides 104 milliseconds of data for each collision measured. This comprises 3-orthogonal channels for the linear accelerometer (H3LIS331DL, STMicroelectronics, Genova, Switzerland, ±200 g, 12-bit resolution) and gyroscope (LSM9DS1, STMicroelectronics, Genova, Switzerland ±35 rad/s, 12-bit resolution), both sampling at 1000 Hz. It should be noted that the illustrated gyroscope is capable of sampling at 1600 Hz, but is optionally down-sampled in this embodiment to 1000 Hz.

### Example Data

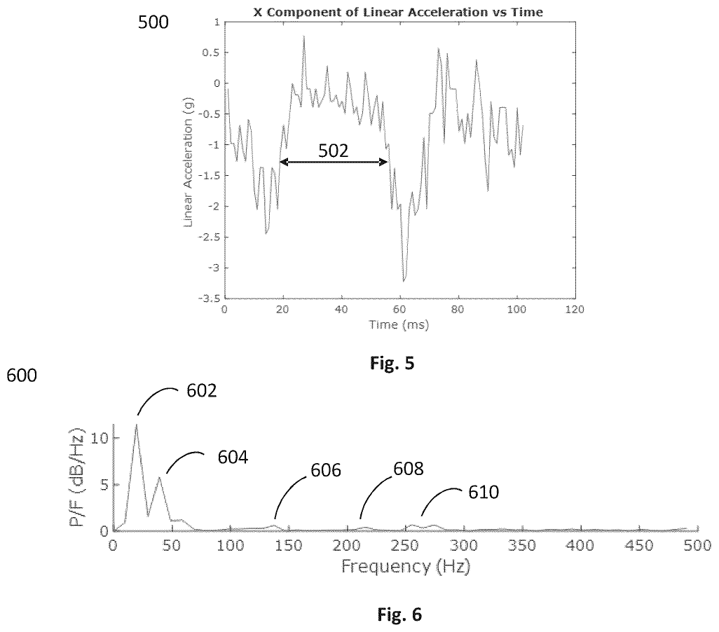

An example of the linear acceleration data that is measured by the system for monitoring acceleration 28 is shown in FIG. 5. FIG. 5 is a line graph 500 with the time in milliseconds along the x-axis and the x-component of the measured linear acceleration in units of g on the y-axis. As can be seen, there are various features that could possibly be identified in this data such as peak position, full width half maximum of any peaks (indicated by double ended arrow 502), and various frequency components (not seen in graph 500). In this example, the peaks could be indicative of an impact received by the wearer of the IMG and each type of impact may have a different representation in the data.

Examples of frequency components can be seen in FIG. 6, which is the acceleration data shown in FIG. 5 in the frequency domain and displayed as a power spectral density graph 600. As will be appreciated, there are two peaks 602 and 604 up to about 50 Hz and small minor peaks 606, 608, and 610 between 100 and 300 Hz. The features identified in both the time domain and the frequency domain can all be used by a trained supervised machine learning model to classify the impacts that are represented by the data.

### High Level Process Overview

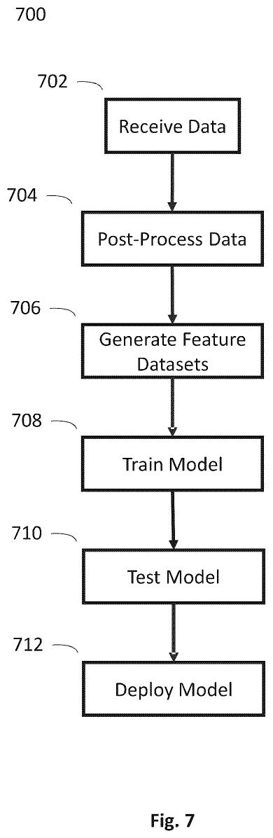

A general outline of a computer implemented method of training a machine learning algorithm for classification of impact data measured by an IMG will now be described with reference to FIG. 7, which is an example process flow control diagram 700. In a first step 702 the linear acceleration data and the rotational angular velocity data (“data”) is received by a processor, such as a processor 34 that may be located in the monitoring station 32. Monitoring station 32 comprises electronic components collectively, individually or in sum or in part providing processing resources for executing the methods of training and operation as described in the following. As described above in relation to FIGS. 1 to 4, the linear acceleration data and the rotational angular velocity data is measured by an IMG, such as described in relation to FIGS. 1 and 2, and, as with all physical measurements, this data will include an element of error (sometimes referred to in the description as noise). The error and/or noise could arise from instrumental, environmental, procedural, and human effects as is known well in the art of measurement.

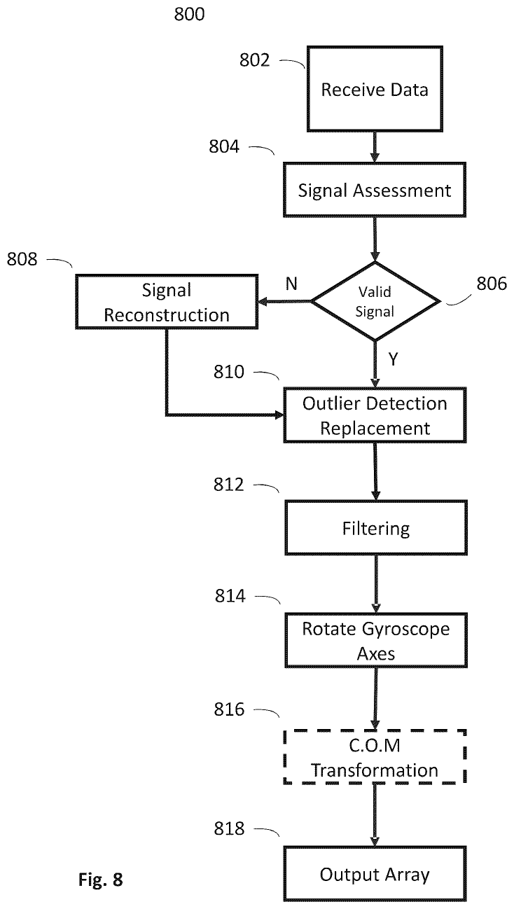

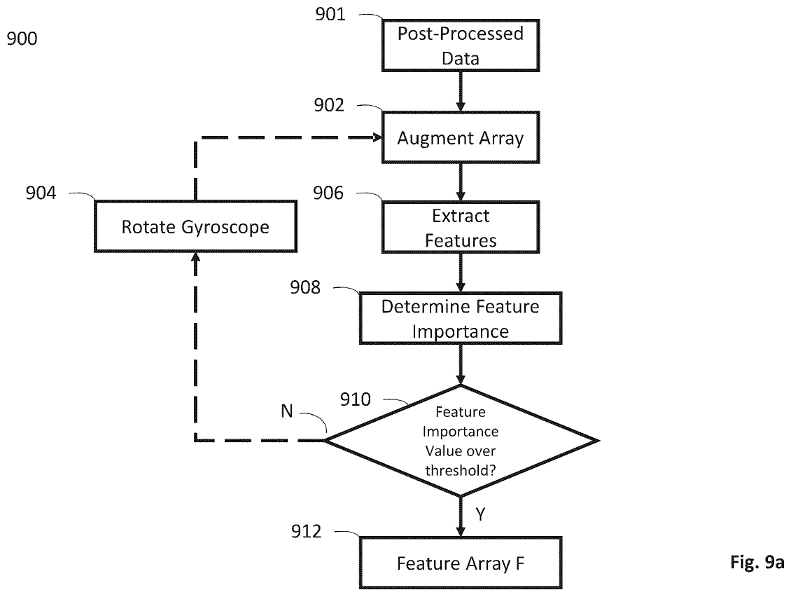

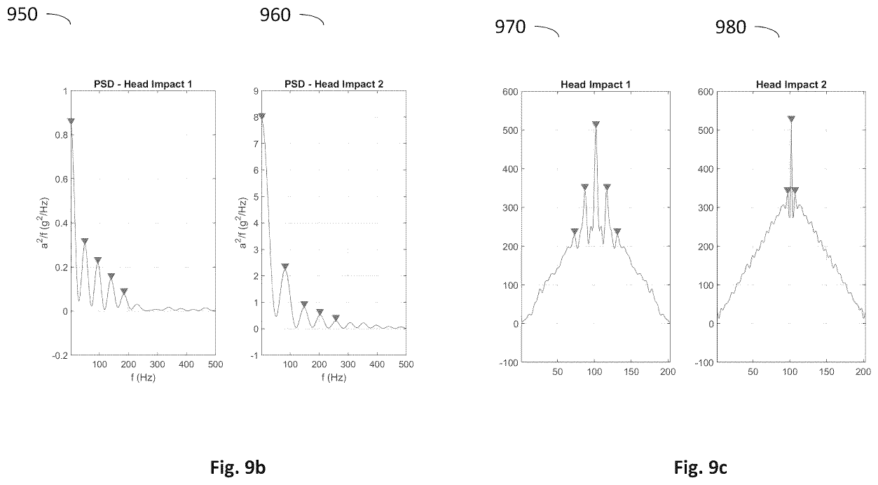

Thus, the data received from the IMG is processed (pre-processed) at step 704 so as to characterise the noise and minimize its effects on the ‘pure’ measurement represented by the data. The processing of the data generates a dataset that can be used for training of a machine learning classifier. Prior to processing, the data used in its “raw” or unprocessed format does not result in accurate results when used for supervised training of a classifier. The processing of the received data and generation of the training data will be described in more detail with reference to FIG. 8 and FIG. 9a respectively.

Once the data has been processed at step 704, the next step is to select time domain features and frequency domain features from the data 706 to generate a training dataset. Examples of such features were described above in relation to FIGS. 5 and 6. In a broad outline, this involves the generation of datasets that can maximise the number of features that can be used to classify the impacts received; converting to a feature array; and then calculating a predictor importance value for the elements in the feature array. This will be described in more detail in relation to FIG. 9a.

Once the features of the dataset have been identified, the next step is to select and train a supervised classifier model 708 using the previously generated training dataset in step 706. This may include the sub-steps of partitioning the data to cross validate the data; selection of a supervised classifier model from a suite of supervised classifiers; and optimization of hyperparameters to obtain a trained supervised classifier model using a first portion of the dataset that has been partitioned.

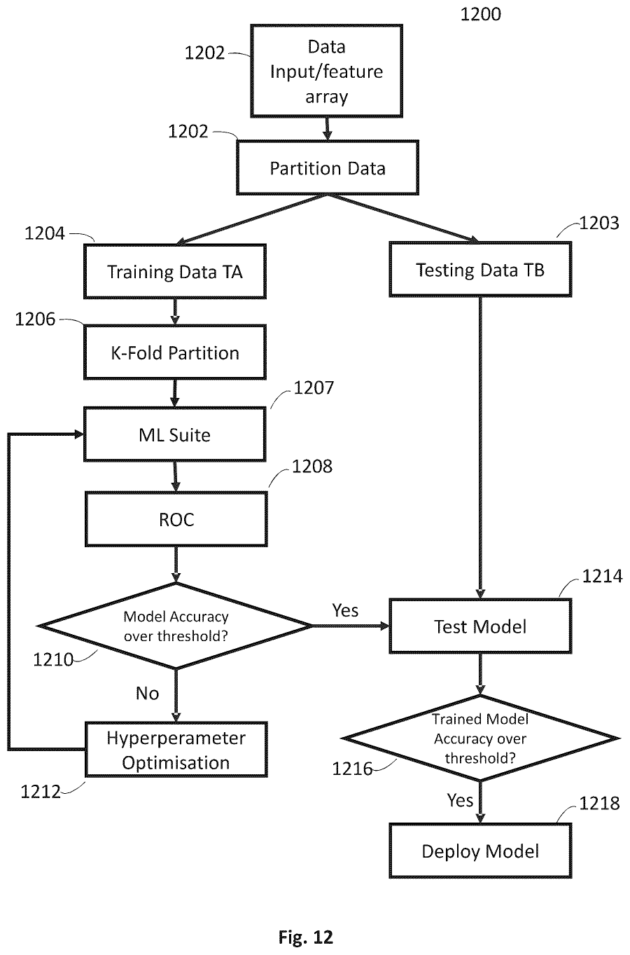

Finally, at step 710, the data can be tested against a second portion of the dataset that was previously partitioned “test dataset”, but not used in the training. The test dataset includes the features and labels for said features to characterise the accuracy of the trained classifier. If the trained supervised classifier model meets user defined criteria, then the model can be deployed at step 712. The training and testing of the supervised classifier model will be described in relation to FIG. 12.

### Processing

The previously introduced process flow step 704 will be described in more detail in relation to FIG. 8. FIG. 8 is a process flow control diagram illustrating an example data process flow 800 according to an embodiment of the invention. During measurement of a physical quantity, in most instances, what is actually measured is a linear combination of the desired ‘idealised’ signal and noise:

$${y}_{M} ( t ) = y ( t ) + η ( t )$$

Here y_{M}(t), represents the discrete measurement of the signal at time t, and y(t) and η(t) are the idealised signal and the imposed error to that signal at time t respectively. The idealised measurement is the true signal if there were no error sources present during the measurement; the error term η(t) can have many sources, for example, be intrinsic to the measurement, system noise, or may be influenced by the surrounding environment. The objective of post-processing signal filtering is to find a balance that minimises η(t), without corrupting y(t).

In the first step 802, linear acceleration and angular velocity data is taken directly from the measurement sensors in the system for monitoring acceleration 28 (that is the linear accelerometer and the gyroscope). As previously mentioned, the data for a single impact event, consists of 100 measurements, for each of the 3 orthogonal axes of the accelerometer and gyroscope respectively. This is sometimes referred to as an “impact” or “impact event”. Of course, other measurement windows may be used resulting in more or less measurements taken per impact, 100 is merely an example.

The received data is stored as a numeric array, A, of size l×m×n, where l=100 and refers to 100 measurements at a frequency of 1000 Hz, m=6, refers to the 6 measurement channels (the first three and latter three columns store measurements recorded by the x, y, z channels of the linear accelerometer and gyroscope respectively) and n is the number of impacts recorded.

$$A = [ \begin{matrix}{a}_{x} ( {t}_{1} ) & {a}_{y} ( {t}_{1} ) & {a}_{z} ( {t}_{1} ) & {ω}_{x} ( {t}_{1} ) & {ω}_{y} ( {t}_{1} ) & {ω}_{z} ( {t}_{1} ) \\ {a}_{x} ( {t}_{2} ) & {a}_{y} ( {t}_{2} ) & {a}_{z} ( {t}_{2} ) & {ω}_{x} ( {t}_{2} ) & {ω}_{y} ( {t}_{2} ) & {ω}_{z} ( {t}_{2} ) \\ ⋮ & ⋮ & ⋮ & ⋮ & ⋮ & ⋮ \\ {a}_{x} ( {t}_{m} ) & {a}_{y} ( {t}_{m} ) & {a}_{z} ( {t}_{m} ) & {ω}_{x} ( {t}_{m} ) & {ω}_{y} ( {t}_{m} ) & {ω}_{z} ( {t}_{m} )\end{matrix} ]$$

At process flow step 804, the data stored in array A is assessed to quantify the amount of measurement data returned by the sensors. Typically, the signal data is transferred from the IMG 10 to an external device in signal packets. Each packet contains 8 measurements per channel. The external device (not shown in the drawings) could be a computer, laptop, data processor, or other device separate to the IMG 10. The data is transmitted from the IMG 10 to the external device for processing. The specific detail of the transmission is not described here. If a packet is not sent or not receivable, i.e., corrupted during measurement or transmission, values within that packet are returned as null (not numerical data). If the number of null measurements per impact per channel, is greater than 24 (3 missed packets, irrespective of position within total signal) the impact is disregarded from the process 800 at step 806.

If the data is disregarded at step 806, the next step 808 of the data processing flow 800 is to reconstruct any signal missing from each impact. Due to the IMG 10 wake up regime, there may be sections of the impact that are not recorded or sent to the processor. Part of the post-processing 800 is to mathematically reconstruct the missed data measured. This is described in relation to pending patent application having attorney reference number P00551874 GB, the description of which incorporated herein by reference. If the data is not disregarded at step 806, then the process flow moves on to step 810.

The post-processing step 810 assesses if there are measurement artifacts or outliers within each of the m signal channels. If there are outliers identified, these measurement points are replaced using an appropriate interpolation scheme. For example, spurious data points can be identified and removed using a Hampel filter, or any other suitable filter. This procedure in step 810 is conducted across all measurement channels for both sensors, thus array A is replaced with array B, A→B.

In the previous process flow step 810, extreme high frequency, high magnitude noise artifacts were removed from the dataset. The next step 812 in the post-processing regime 800, utilises an active filter to further remove moderate to high frequency noise features from the measured signals. The sinusoidal noise is removed from the time series data, which is periodic signals which can be approximated by a sum of sinusoids.

The post-processing regime 800 proceeds to step 814 which rotates the gyroscope axes to match that to the linear accelerometer axes. The reason for this step is that within the IMG, the x, y, and z axes of the gyroscope are not aligned with the x, y, and z axes of the linear accelerometer respectively. The reason the axes are not aligned is because the gyroscope and accelerometer are separate devices in different locations within the IMG 10. They are placed separately and so it is not impossible that they both have different relative locations and orientations between them. This different orientation is often different from IMG to IMG and so there is no single or uniform calibration for the IMGs. Without doing this, the differing orientation may result in difficulties using the two data sources for use as training a machine learning model classifier.

The next optional step is a centre of mass transformation 816. The IMG is not at the centre of gravity of the user, which means that during rotation an acceleration may be recorded even though the centre of mass is not accelerating. The transformation to centre of gravity 816 corrects this and does so with standard rigid body transformations. This has the benefit of selecting features for classification from a ‘truer’ measurement.

At step 818 the input data, array B, is replaced with array C, B→C and outputted.

At this point in the supervised training method, the data has been assessed as to whether or not it can be used for the training, and it has undergone various post-processing steps described above. The feature generation step 706 will now be described in more detail with reference to FIGS. 9a to 11.

### Feature Generation

FIG. 9a is a process flow control diagram 900 according to an embodiment of the invention. The input 901 to this process is array C, which contains the post-processed measurements of the linear accelerometer and gyroscope as described in relation to FIG. 8 above. The first 3 columns of C correspond to the three orthogonal components of linear acceleration, a(t)=(aₓ(t), a_{y}(t), a_{z}(t)) and similarly the last three columns of C correspond to the three orthogonal components of the rotated angular velocity, ω(t)=(ωₓ(t), ω_{y}(t), ω_{z}(t), where t ranges from 1 to 100 ms, thus, the data generated per impact event is m×n=600 datapoints. For comparison, a standard image classifier would take 64×64 pixel images (or 4096 datapoints) for feature extraction, analysis, and categorisation.

Therefore, to enhance the identification of potential candidate features with high importance scores, the impact event dataset is augmented by additional values a(t) and ω(t) to generate additional auxiliary vectors at process flow control step 902. These auxiliary vectors are evaluated using mathematical operations or using algebraic combinations of the original or auxiliary vectors to generate further auxiliary vectors.

As an example, a(t) could be numerically differentiated to yield the corresponding jerk, j(t), or numerically integrated to yield the velocity v(t), or double integrated to yield the displacement d(t) vectors. Similarly, ω(t) could be differentiated to obtain the angular acceleration, a(t). In the latter case, both original and auxiliary vectors can then be combined and compiled into an array, D where

$$D = [ a ( t ) , ω ( t ) , j ( t ) , α ( t ) , \frac{ω ( t )}{a ( t )} , \frac{α ( t )}{a ( t )} , ... , \frac{ω ( t )}{v ( t )} , ... , \frac{α ( t )}{d ( t )} ] ,$$

in order to enhance the number of datapoints per impact, i.e., 100×3×number of vectors in D. The reason for the specific choice of mathematical operations referred to above in describing the illustrative embodiment is because they represent a physical variable, i.e., integrating acceleration with respect to time to produce the velocity. It should be appreciated that any combination or mathematical operation could be used irrespective of whether it represents a common physical variable.

The next process control step, 906, takes the augmented data of array D and extracts time-domain features and frequency domain features from the augmented data set. The result of process flow step 906 is to produce an array of measurement data having fewer data points than contained in array D. The result of the process flow step of the feature extraction process 900 is to convert the augmented measurement data in array D to a feature array F. Table 1 is a non-exhaustive list of the features that may be extracted from array D each data column, y (y being a 100-element data array), for one or more embodiments in accordance with the present invention:

TABLE 1
a list of functions applied to y(t) to generate classification features

| Name | Data array y(t) |
|---|---|
| Minimum | yₘᵢₙ = min\|y\| |
| Maximum | yₘₐₓ = max\|y\| |
| Mean | $μ = \frac{1}{N} {\sum }_{i = 1}^{N} {y}_{i}$ |
| Standard Deviation | $σ = \sqrt{\frac{1}{N - 1} {\sum }_{i = 1}^{N} {\vert {y}_{i} - μ \vert}^{2}}$ |
| Root-Mean-Square value | ${y}_{RMS} = \sqrt{\frac{1}{N} {\sum }_{i = 1}^{N} {\vert {y}_{i} \vert}^{2}}$ |
| Mean Absolute Deviation | ${y}_{MAD} = \frac{1}{N} {\sum }_{i = 1}^{N} \vert {y}_{i} - μ \vert$ |
| Skewness | $s = \frac{{E ( y - μ )}^{3}}{{σ}^{3}}$ |
| Kurtosis | $k = \frac{{E ( y - μ )}^{4}}{{σ}^{4}}$ |
| Autocorrelation/ cross-correlation | ${R}_{xy} ( m ) = \{ \begin{matrix}{\sum }_{n = 0}^{N - m - 1} {x}_{n + y} {y}_{n} , & m \geq 0 , \\ {R}_{xy} ( - m ) & m < 0.\end{matrix}$ |
| Spectral Power, peak magnitude, and position | $P ( f ) = \frac{Δ t}{N} {\vert {\sum }_{n = 0}^{N - 1} {x}_{n} {e}^{- i 2 π f Δ tn} \vert}^{2} , - \frac{1}{2 Δ t} < f < \frac{1}{2 Δ t}$ |

The number of rows in array F corresponds to the number of impact events measured, M, and the number of columns, N, refers to the number of features extracted per impact from the array D. Thus, F has the form:

$$F = [ \begin{matrix}{f}_{1}^{1} & {f}_{2}^{1} & … & {f}_{N}^{1} \\ {f}_{1}^{2} & {f}_{2}^{2} & … & {f}_{N}^{2} \\ ⋮ & ⋮ & … & ⋮ \\ {f}_{1}^{M} & {f}_{2}^{M} & … & {f}_{N}^{M}\end{matrix} ]$$

For example, the first row of feature array F may be features extracted from a time series data of an impact event which is classified as an “ObliqueFrontRight” impact. The second row would be from a separate impact event and so on. Examples of some of the possible classification features are shown in FIG. 9b and FIG. 9c. FIG. 9b shows Welch power spectral density (PSD) graphs for the x component of linear acceleration for two separate direct head impacts 950 and 960. The autocorrelations of the same two impacts are shown in plots 970 and 980 of FIG. 9c. One can see that the PSD for the two different head impacts produce a frequency comb which have different peak positions and peak magnitudes. The positions of the peaks in each frequency comb may be characteristic of that type of impact. In the graphs 950, 960, the peaks of the frequency combs are shown with triangle markers. One can see that there appears to be some correspondence between the two PSD graphs for the two similar impacts. For example, the overall ‘shape’ of the two plots appears to be similar. Of course, the peak values and positions of each peak or sub peak may be different between the two, but there may be some underlying similarity that can be identified for the purposes of automatically classifying the type of impact. Instead of the absolute peak magnitude being used as a classification feature, the relative magnitude may also be used.

The autocorrelation classification feature from table 1 above is shown in FIG. 9c for two different head impacts, plots 970 and 980. The autocorrelation plots 970 and 980 could also be said to be similar in that they qualitatively appear to have the same form and shape. It can be seen that even though the peak position and relative intensity of the peaks are different, the relative values may be used as a classification feature data point for feature array F, thus reducing the need for actual measurement data in array D to fewer points than would otherwise be necessary.

Returning to FIG. 9a, the next step, 908, of the process flow control diagram 900 calculates the predictor importance of all N elements of the feature array F, for all M realisations against a response array, R. The predictor importance value is the relative importance of each feature in a row of array F in classifying impacts. High values of predictor importance indicates that the particular feature is more important for classifying the type of impact that generated the feature. For example, it might conceived that the standard deviation of the data for a given impact is found to be very good for identifying the type of impact, and therefore, it would be given a high predictor importance value.

In order to calculate the predictor importance value, a response array is used. The response array R, is an M×1 array whose elements label each feature of a row of F with a numeric or category classification. For example, the set of responses in rugby union, may refer to the type of impact observed and classified via video analysis or human analysis (not described in detail here). Some examples of post-analysis impact identification protocols cover impact type, anatomical position, and collision outcome.

An illustrative representation of a response array R showing the elements as impact type is shown below:

$$R = [ \begin{matrix}{r}_{1}^{1} = ObliqueFrontRight \\ {r}_{1}^{2} = Back \\ {r}_{1}^{3} = Facemask\end{matrix} ]$$

This means that the features from feature array F in row 1, i.e. F_{N}¹=[f₁¹ f₂¹ . . . f_{N}¹] correspond to the impact label in response array R of r₁¹ which in the described embodiment is an “ObliqueFrontRight” impact; the second row is classified as “Back” and third row as a “Facemask” impact. As mentioned above, the response array R may also contain other numeric or categorical classifications.

The predictor importance of a feature of a row of array F is determined using the minimum redundancy maximum relevance (MRMR) algorithm, an example of which may be found in reference [1]. Application of the MRMR algorithm to the elements of vector F identifies a subset of its features that are mutually and maximally dissimilar for the classification of the impacts. The algorithm minimizes, or at least reduces, the redundancy of a feature set and maximizes, or at least increases, the relevance of a feature set to the response variable (i.e. the classification of the impact). The term “redundancy” is used here to refer to features in the feature array that correlate with other features in the feature array. The best descriptors of the data will be independent from other descriptors of the data. Moreover, the algorithm quantifies the redundancy and relevance using the mutual information of variables—pairwise mutual information of features and mutual information of a feature and the response.

In the next step of the process flow 908, the MRMR algorithm returns Λ, a 1×N array, whose elements represent the relative importance of the corresponding feature of a row of array F. Typically, q elements with the greatest magnitude from within Λ are identified and stored within a new array G. In this example the MRMR identified >50 features with a predictor importance score >0.1.

At process flow step 910 the feature importance values that were previously determined in step 908 are compared to predetermined threshold. If they are greater than the predetermined threshold, then the process flow 900 moves to step 912 which will be described below. If there are no predictor importance values meeting the predetermined threshold, or fewer than desired, then the process flow 900 moves to optional rotation step 904. The reason it is shown in dashed lines indicate that if condition 910 is satisfied, then it does not proceed to optional step 904.

### Optional Rotation

In process flow control diagram step 904, the measurement data array D may optionally be further extended and augmented through the inclusion of rotated versions and derived/integrated forms of the rotated angular velocity, ω(t). In the array D, the measurement directions are parallel to the sensor axis, whereas the latter case indicates that the measured rotations are around each orthogonal axis. Despite the difference in measurement direction between the two sensors, some features may be strongly correlated, i.e., collinear, and as such contain similar information about the variance of the features for a given impact, e.g., position of peak values. To minimise collinearity of features between the components and derived/integrated forms of a(t) and ω(t), the coordinate axes of the gyroscope is iteratively rotated with respect to the linear accelerometer to minimise collinearity. Thus, the array D is optionally extended to the array E, which includes rotated versions of the original, auxiliary, and combined vectors, i.e., E=[D, ω^{αβγ}(t), . . . ], where the α, β, and γ superscripts refer to the Euler angles of rotation applied to the x, y, and z-components respectively of ω(t) and its derived forms. The rotation of data is an improved step of pre-processing data. The feature generation process flow 900 is finally iterated through all its sub-components until the array G, has achieved Q>>q columns. Here, the iteration process incrementally alters the magnitude of α, β, and γ with values in the interval

$$[ - \frac{p i}{4} \frac{p i}{4} ] .$$

Due to manufacturing tolerances and the variations of the shape of the instrumented IMGs to suit dimensions of an intended user, the relative position of the axis of the gyroscope and accelerometer can vary between different IMGs. Therefore, there is no ‘one size fits all’ calibration that can be applied to pre-process the data for use in the supervised training of a classifier. The method of iteratively rotating the gyroscope axes relative to the accelerometer described above provides a method of generating a training dataset that has been customised depending on the specific spatial positioning of the sensors in the hardware. To the extent that the specific spatial positioning is dependent upon the intended user, the training dataset may be regarded as personalised to the intended user of the IMG. The data measured between IMGs will be pre-processed differently depending on the arrangement of the sensors within the IMG, which is a bespoke solution for each IMG. This means that a specific calibration technique is unnecessary in the manufacturing process of the instrumented IMG and thus reduces costs of the device to a consumer. The preprocessing method is flexible and does not require a specific form of data to be input into it.

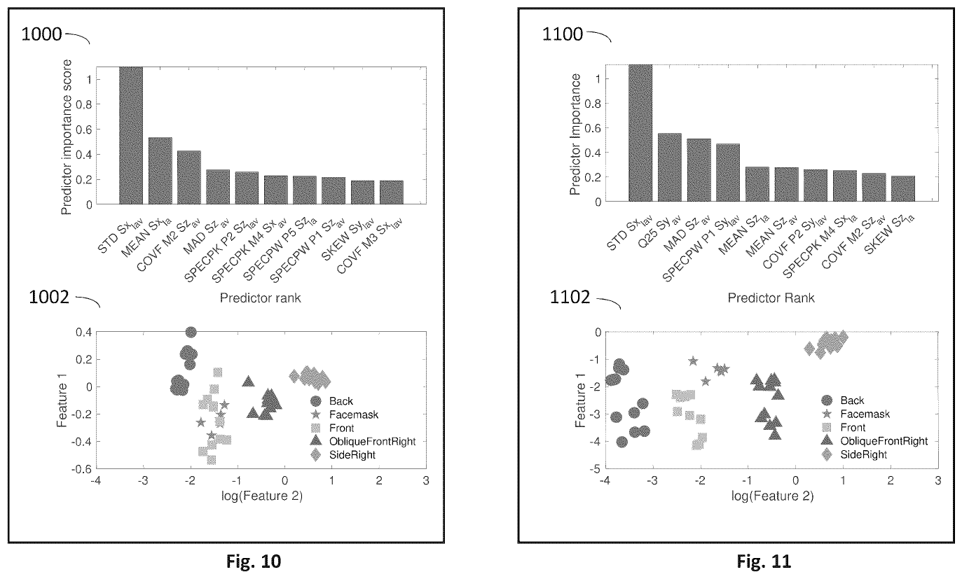

Moreover, due to this rotation technique the training dataset provided to for supervised training of a machine learning classifier is improved because there are better features generated at step 906, as evidenced by the data described below in relation to FIGS. 10, 11, 12, and 13.

The rotated gyroscope data combined with the augmentation step at 902 increases the available data to be used for feature generation. This improves the features generated and thus improves the training dataset which in turn will improve the accuracy of the trained classifier as explained in more detail below.

### Rotated Data Examples

An illustrative example of the outcome of the gyroscope rotation process 904 is described in relation to FIG. 10 and FIG. 11. The plots 1000 and 1100 are histograms of predictor importance rank (q=10) as a function of feature, the lefthand with zero rotation (α=β=γ=0) the righthand with α=5°, β=γ=0 (after step 902 described above). That is, q=10 meaning that the 10 highest ranked features are shown. In both cases the MRMR algorithm has identified the standard deviation of the signal's x-component as the highest ranked predictor, where the subscript of the feature (lav) refers to the auxiliary dataset

$$\frac{ω ( t )}{a ( t )} .$$

The plot 1002 and the plot 1102 displays the ranked feature (q=1) plotted against ranked feature (q=2), the left with zero rotation (α=β=γ=0) and the right with α=5°, β=γ=0. In each case, the response groups are identified with different shaped markers. With no rotation of the gyroscope data, the second ranked feature (q=2) is the mean position of the x-component of a(t), the response variables ‘Facemask’ and ‘Front’ occupy overlapping positions in feature space. However, when the gyroscope axis has been rotated, the second ranked feature (q=2) is now the lower quartile of the y-component of ω(t), and all response variables occupy distinct regions in the feature space.

The final step of process flow 900 is to output the feature array at step 912.

### Supervised Training

As described above, once the classification features are identified, the next step, 708, (from FIG. 7) is the generation of a supervised classifier model that maximises the correlation of the feature vectors complied in array G, with their corresponding response, i.e., the elements within the previously described response vector R. This step will now be described with reference to FIGS. 12 to 16.

FIG. 12 shows an illustrative process flow diagram 1200 for the selection, generation, and optimisation of a supervised classifier model (also called the training process flow 1200). The first step 1201 of the training process flow is to receive the feature array G and corresponding response array R.

The next step 1202 of the training process flow is to partition the aforementioned feature array G and response vector R, into a training dataset 1204 and a testing dataset 1203. Only the training dataset 1204 is used for selection and training of the supervised machine learning classification algorithm; the testing dataset is held for testing a selected supervised classifier after training. The partitioning ratio for both datasets is defined, for example, as 75:25, where 75% of the corresponding rows of arrays G and R are assigned to the training dataset 1204 and the associated response array R. Similarly, 25% of array G and corresponding responses in array R are assigned to the testing dataset 1206. A selection of rows based on the portioning ratio is conducted randomly. Of course, other partition ratios and criteria may be used depending on the requirements.

Once partitioned, the next step 1206 sets the cross-validation scheme, which further partitions the training dataset 1204 into k-folds. K-fold cross validation is used to compare and select a supervised classifier model for a given predictive modelling problem because it is simple to understand and implement, and results in skill estimates that generally have a lower bias than other methods. K-fold cross validation is a method of removing overfitting/bias when modelling. Where the average values from the different folds are used to summarise the performance of the model.

K-folds are subsets of the training data, which are used as a hold out test and the remaining training data is evaluated against it, with the results of the model retained. In this embodiment, a 5-fold cross-validation partition is utilised, where each fold, has approximately the same number of observations and contains approximately the same number of response/class proportions.

The next step of the process flow is to insert the training dataset 1204 into a suite of supervised classifiers 1207 (“suite 1207”) to calculate the classification accuracy of each row (measurement) of the training dataset 1204.

The suite of supervised classifiers 1207 and their corresponding hyperparameters used for the described embodiment are shown in Table 2 below.

TABLE 2
Non-exhaustive list of supervised classifiers
and their corresponding hyperparameters.

| Cⁱ | Classifier Algorithm (1206) | Hyperparameters (h) |
|---|---|---|
| 1 | Decision Trees | Maximum number of splits |
| | | Split criterion |
| 2 | Support Vector Machines | Kernel function |
| | | Box constraint level |
| | | Multiclass method |
| 3 | Ensembles | Method - e.g., AdaBoost |
| | | Learner type - e.g., Decision Trees |
| | | Number of learners |
| | | Learning rate |
| 4 | Neural Nets | Number of connected layers |
| | | Activation |
| | | Regularization strength |
| 5 | Naïve Bayes | Distribution type |
| | | Kernel function |
| 6 | Nearest Neighbours | Number of neighbours |
| | | Distance metric and weight |

The classifiers of the suite of supervised classifiers 1206 are generically represented in the following form:

y=Cⁱ(X,x,h)

where Cⁱ denotes the type of classifier. In the example of table 2 above, i=1 implies the classifier is the decision tree. X is the training feature array (in this instance, training dataset 1204 described above), x is the training response vector (the partition of array R that is used for training) and h a vector containing hyperparameter initial values. Given an initial input X, x and h all classifiers return a value y, which contains the true positive and false negative rates for the given inputs. Referring back to FIG. 12, at step 1208 these true positive and false negative values are used to generate confusion matrices and receiver operating characteristic (ROC) curves to quantify performance of each classifier. The ROC provides a measure of the selectivity and sensitivity of the generated model by depicting the true positive rate (TPR) with respect to the false positive rate (FPR).

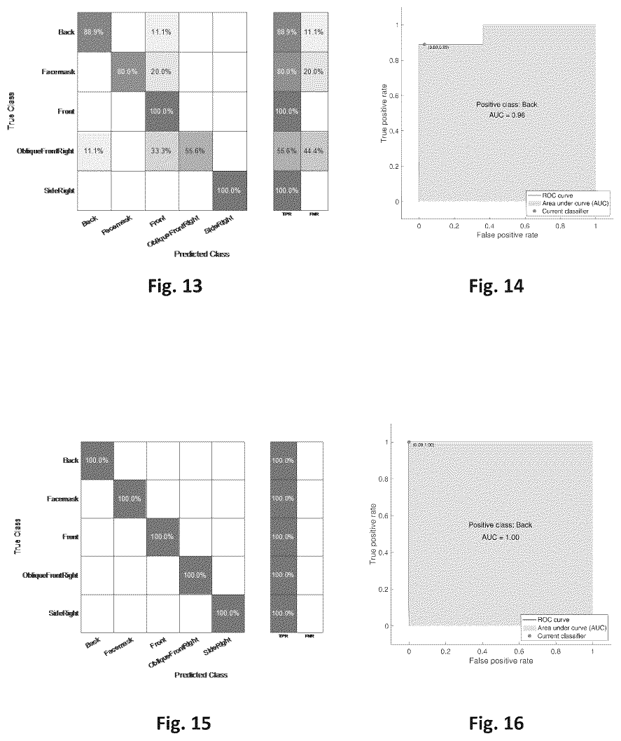

An illustrative example of a confusion matrix and ROC curve is shown in FIG. 13 and FIG. 14 respectively. FIG. 13 is an illustrative example confusion matrix that is output from a linear discriminant classifier (not shown in table 2) which was given the inputs X, x and h. The TPR and false negative rate (FNR) on the righthand-side of the confusion matrix displays the percentage accuracy of classified and mis-classified impacts by this classifier for the TA dataset. On the graph of FIG. 13 (lefthand-side of FIG. 13), the predicted impact classification is shown on the x-axis versus the actual impact classification on the y-axis and the percentage of predicted classifications is indicated in the corresponding boxes. For example, the response element ‘Front’ and ‘SideRight’ impacts were correctly classified because the TPR is 100%, which is indicated in the confusion matrix by the value “100%” in the ‘Front’ and ‘SideRight’ columns on both of the x-axis and y-axis respectively. The other three elements, ‘FaceMask’, ‘ObliqueFrontRright’, and ‘Back’ were mis-classified, which is also indicated in the righthand-side of the figure. The discriminant classifier predicted the ‘ObliqueFrontRight’ impact correctly 55.6% of the time, and incorrectly as ‘Back’ and ‘Front’ 11.1% and 33.3% of the time respectively.

FIG. 14 displays the ROC curve, which is generated by plotting the TPR as a function of FPR, and also indicated is the area under the curve (AUC), numerically deduced to be 0.96. The overall goal of this training process (i.e., the combined hyperparameter and axis rotation optimisation procedures), is to maximise both the ROC and AUC scores. That is to maximise correct impact classification given a response vector R.

To calculate the classification accuracy, the calculated classification of the supervised classifiers from the suite 1207 is compared to the actual response in response array R to determine true positive and false negative rates.

As mentioned, at step 1208 the parameters TPR and FPR are then utilised to calculate the receiver operating characteristic (ROC) of the best performing classification algorithm (BPCA).

At step 1210, the computer implemented method 1200 determines if the ROC/AUC fulfils user-defined accuracy thresholds by comparing the ROC and the AUC scores to a pre-defined acceptance value. Typically, ROC and AUC scores greater than 0.85 are accepted, which is due to the inherent variability of measured raw measurement data (array A) given the same response. However, in many cases like that shown in FIG. 13 and FIG. 14, these performance metrics can be improved via both optimisation procedures.

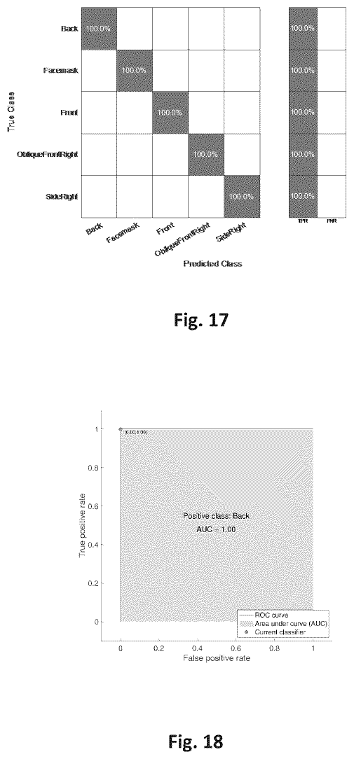

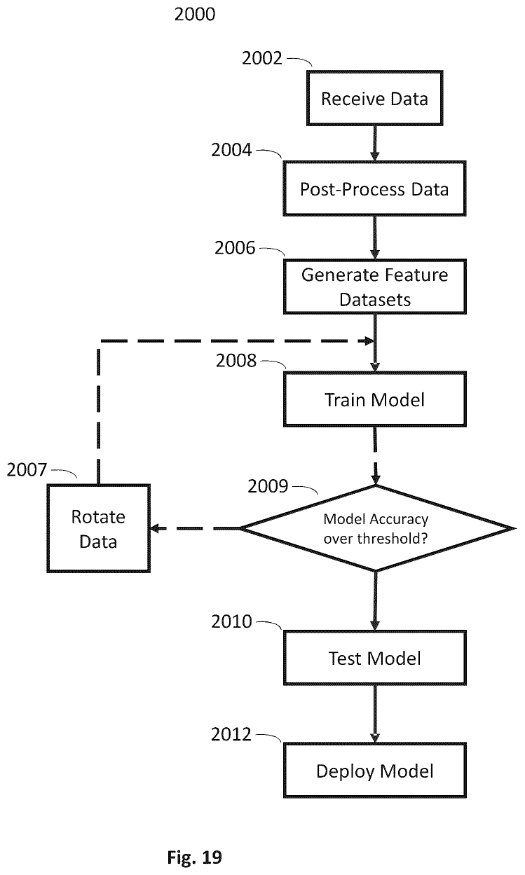

If the ROC/AUC meets the predefined criterion, then the model is passed at step 1214 to be tested. In this embodiment, a value of 0.8 is used. The model testing step 1214 receives the testing dataset 1203 and uses the approved/discovered classifier model from steps 1204 to 1210 to access the accuracy of the model (1216). The predicted response of the trained classifier model is then compared against the true response analogous to that implemented during training (steps 1207-1210). FIG. 17 and FIG. 18 show the results of the trained classifier being used with the test dataset 1203. FIG. 17 is a confusion matrix that is similar to as those described above in relation to FIG. 13 and FIG. 15. FIG. 18 is an ROC curve that is similar to as was described in relation to FIG. 14 and FIG. 16. FIG. 17 and FIG. 18 show that the trained classifier was able to correctly classify the impact types with 100% accuracy and an AUC of 1 as a result of the optimisations described above.

Finally, the model is deployed at step 1218. The deployment can either be as a numerical function or an executable file. The former can be Matlab, Python, or C, and can have both training and testing datasets embedded for further testing or refinement. Similarly, the model can be packaged as an executable, again with the option to embed the training and testing datasets.

Returning now to FIG. 12, if at step 1210 the classification accuracy is not sufficient and does not meet the pre-defined criteria, then the hyperparameters of the initial top performing classifier, such as those in the righthand column of table 2, are iteratively refined to maximise performance at step 1212. The optimised hyperparameter classifier is then tested again at step 1210 and the hyperparameters are further optimised and reinserted until it meets the criteria set out in 1210.

FIG. 15 is a confusion matrix generated from an Ensemble classifier after hyperparameter optimisation at step 1212, and it can be seen that all impacts are classified correctly. Accordingly, calculated AUC from the ROC curve of FIG. 16 has a value of 1 indicating that the classification is 100% correct for the training portion of the dataset 1204. The numerical procedure utilised Bayesian optimisation to refine the hyperparameters, h, of Cⁱ(X, x, h). However, other optimisation regimes may be used such as Random Search and Grid Optimisation.

### Overarching Rotation Optimisation

Referring back to FIG. 12, if at step 1210, following hyperparameter optimisation, the ROC and AUC is still below a user-defined threshold, then the input Array G is rotated as described in optional step 902 of FIG. 9. This is shown in FIG. 20, which is very similar to FIG. 12 with like processes being represented by the same reference numeral except incremented by 800 (for example the “receive data A(t)” step 702 is the same as 2002). As described above, the only difference is the additional step 2009 which breaks the hyperparameter testing iterative loop (steps 1207, 1208, 1210) to rotate the gyroscope axes as described in optional step 902 of FIG. 9 until a classifier is discovered and trained to achieve an acceptable ROC and/or AUC. Typically, ROC and/or AUC values >0.85 is accepted.

This rotation step is similar to that described before with reference to process step 902 in that it provides a customisable solution to the problem that the absolute position of the two sensors in the IMG is not known. The iterative rotation the axes of the gyroscope compensates for variable sensor positioning and orientation, such as might be the case with relatively low cost, “off the shelf” devices. This allows such devices to be used to accurately characterise the type of impact without the need for expensive equipment or calibration. The use of simple technology can be used.

The method of automatically classifying the impacts reduces if not removes the need for spotters on a sports pitch and provides quantitative data which can be used by off pitch personnel to access if the player needs medical attention or the like.

At process flow step 902 example mathematical operations on the acceleration data and the angular velocity data received from the IMG that results in other physical variables is disclosed. However, the method is not strictly to be limited to mathematical operations that result in vectors representing physical variables (such as integrating acceleration to result in velocity). Other mathematical operations on the vectors representing the physical measured data may be envisaged.

Additionally, all references made herein to orientation (e.g. front, rear, upper, lower, anterior and posterior) are made for the purposes of describing relative spatial arrangements of features, and are not intended to be limiting in any sense.

It will be understood by those skilled in the art that the drawings are merely diagrammatic and that further items of equipment may be required in a commercial apparatus. The position of such ancillary items of equipment forms no part of the present invention and is in accordance with conventional practice in the art.

Insofar as embodiments of the invention described above are implementable, at least in part, using a software-controlled programmable processing device such as a general purpose processor or special-purposes processor, digital signal processor, microprocessor, or other processing device, data processing apparatus or computer system it will be appreciated that a computer program for configuring a programmable device, apparatus or system to implement methods and apparatus is envisaged as an aspect of the present invention. It will also be understood that in the IMG and monitoring system there will be various electronic components that collectively, individually or in sum or in part provide processing resources for the IMG 10 and or the monitoring system. The computer program may be embodied as any suitable type of code, such as source code, object code, compiled code, interpreted code, executable code, static code, dynamic code, and the like. The instructions may be implemented using any suitable high-level, low-level, object-oriented, visual, compiled and/or interpreted programming language, such as, Liberate, OCAP, MHP, Flash, HTML and associated languages, JavaScript, PHP, C, C++, Python, Nodejs, Java, BASIC, Perl, Matlab, Pascal, Visual BASIC, ActiveX, assembly language, machine code, and so forth. A skilled person would readily understand that term “computer” in its most general sense encompasses programmable devices such as referred to above, and data processing apparatus and computer systems.

Suitably, the computer program is stored on a carrier medium in machine readable form, for example the carrier medium may comprise memory, removable or non-removable media, erasable or non-erasable media, writeable or re-writeable media, digital or analogue media, hard disk, floppy disk, Compact Disk Read Only Memory (CD-ROM), Company Disk Recordable (CD-R), Compact Disk Rewriteable (CD-RW), optical disk, magnetic media, magneto-optical media, removable memory cards or disks, various types of Digital Versatile Disk (DVD) subscriber identity module, tape, cassette solid-state memory.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Moreover, one or more features of different embodiments may be combined to create further embodiments not specifically described herein, and any one or more features may be combined consistent with their technical and operational compatibility. To the extent that one or more features from respective embodiments may not be combined without being inconsistent with the technical and/or operational compatibility such features from respective embodiments may be selected for combination which do not have such technical and/or operational compatibility. All such embodiments are contemplated within the scope of this disclosure.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the invention. This is done merely for convenience and to give a general sense of the invention. This description should be read to include one or at least one and the singular also includes the plural unless it is obvious that it is meant otherwise.

In view of the foregoing description, it will be evident to a person skilled in the art that various modifications may be made within the scope of the invention.

For example, although embodiments have been described in which impact event data may be transmitted to a monitoring station in real-time, impact event data may be stored and downloaded wirelessly, or by wired coupling, at breaks in a match, e.g. half-time, or at the end of the match. This may be particularly suitable for non-professional environments and download may be to a device such as a smartphone or other mobile communication device running a suitable application. Additionally, the impact event data may be processed on the IMG in a monitoring station as described being located on the IMG. This provides a device which can analyse the data to be transmitted off the IMG to a transceiver in a similar monitoring station.

One or more embodiments have been described in the context of acceleration monitoring. Optionally, monitoring circuitry, power sources and transmitter and/or receiver circuitry may be included for monitoring other factors such as, for example, physiological data, for example, hydration, temperature, electrolyte levels, amongst other things. Additionally, angular acceleration may be measure instead of angular velocity. Although a first embodiment has been described utilizing a H3LIS accelerometer, embodiments in accordance with the present invention are not limited to use of such an accelerometer. Additionally, control circuitry has been described implemented as a processor but the processor may comprise more than one processing resource and or discrete circuitry and circuit components.

In the description above, the terms “data”, “raw data”, “acceleration data”, and “signal” are all used interchangeably.

In the description of the embodiments of the invention and figures above, the improved training regime is described in relation to classification of impacts that have been measured by an IMG. However, the preprocessing of the data received from the IMG and generation of the training data can equally be used for other applications such as processing of sound waves for voice recognition.

Although an embodiment in accordance with the present invention has been described with reference to an illustrative suite of supervised classifiers, other suitable supervised classifiers may be used, instead of or in combination with one or more of the illustrated supervised classifiers. Additionally, a reduced set of supervised classifiers may be used selected from the illustrated suite.

1. A computer implemented method of generating a feature array configured for training a classifier of impact data measured by a mouthguard, the method comprising:
a) receiving signal data, the received signal data representative of linear acceleration and rotational velocity measurement time series signals indicative of an impact measured from an instrumented mouthguard;
b) receiving impact classification data representative of feature information that specifies the class of impact of each received signal data and storing the impact classification in a response array;
c) extracting one or more features from the signal data and storing in an array, the one or more features corresponding to a single impact;
d) comparing each extracted feature to the corresponding response array element to select the set of features that respectively satisfy a classification relevance threshold indicative of a classification relevance; and
e) responsive to the number of selected features being less than a feature threshold, iteratively rotating the direction of respective x, y, z components of the rotational velocity time series data relative to corresponding x, y, z components of the linear acceleration time series data and performing steps c) to d) until the number of selected features meet the feature threshold.

2. The computer implemented method according to claim 1, wherein the one or more extracted features are time domain features and/or frequency domain features.

3. The computer implemented method according to claim 1, wherein a further pre-processing step of rotationally aligning respective x, y, z components of the rotational velocity time series data to match that of corresponding x, y, z components of the linear acceleration time series data is performed prior to step c).

4. The computer implemented method according to claim 1, wherein the received data is filtered to remove cyclically occurring noise prior to step c).

5. The computer implemented method according to claim 1, wherein the received signal data comprises one or more discrete signal data packets, and responsive to one or more of the discrete signal data packets comprising no linear acceleration and rotational velocity measurement time series signal data indicative of an impact measured from an instrumented mouthguard, reconstructing the signal data.

6. The computer implemented method according to claim 1, wherein the signal data is reconstructed by interpolation.

7. The computer implemented method according to claim 1, wherein the classification relevance threshold is calculated by a minimum redundancy maximum relevance (MRMR) algorithm.

8. The computer implemented method according to claim 1, wherein the time domain features and/or frequency domain features are one or more of the following: Minimum, Maximum, Mean, Standard Deviation, Root-Mean-Square value, Mean Absolute Deviation, Skewness, Kurtosis, Autocorrelation, cross-correlation, Spectral Power, peak magnitude, and peak position.

9. A computer implemented method of training a machine learning model for classification of impact data measured by a mouthguard comprising:
a) providing the feature dataset and the response array according to claim 1;
b) partitioning the feature dataset into a feature array training data dataset and a feature array test dataset;
c) partitioning the response array into a response array training dataset and a response array test dataset, the elements of the response array training dataset corresponding to the elements of the feature array training dataset and the elements of the response array test dataset corresponding to the feature array test dataset;
d) inserting the feature array training dataset into one or more classifier models and training the selected classifier model using the training dataset;
e) evaluating each classifier model accuracy using the test dataset to determine a value indicative of the correct classifications on the response array training dataset;
f) comparing the classifier model accuracy to a predetermined accuracy; and
g) iteratively updating the hyperparameters of the selected classifier model and performing steps c) to d) until the classifier model accuracy is over the predetermined accuracy.

10. The computer implemented method of training a machine learning algorithm according to claim 9 wherein:
responsive to the model accuracy determined in step g) failing to exceed the predetermined threshold:
h) iteratively rotating one or more of the axes of the rotational velocity time series data relative to the corresponding linear acceleration axes and performing steps c) to g) until the model accuracy determined in step g) attains or exceeds the predetermined accuracy.

11. The computer implemented method of training a machine learning algorithm according to claim 9 wherein the predetermined accuracy is the area under the curve and/or receiver operating characteristic.

12. A computer implemented method of classifying impact data measured by an instrumented mouthguard comprising the steps of:
a) receiving signal data, the received signal data representative of linear acceleration and rotational velocity measurement time series signals indicative of an impact measured from an instrumented mouthguard;
b) providing the trained machine learning model of claim 9; and
c) inserting the received signal data into the trained machine learning model.

13. A computer-readable storage medium containing instructions thereon implementable by a computer to carry out the method of claim 1.

14. A data processing apparatus comprising data processing resources configured to implement the method of claim 1 for generating a feature array configured for training a classifier of impact data measured by a mouthguard.

15. A data processing apparatus, comprising data processing resources configured to implement the method of claim 9 for training a machine learning model for classification of impact data measured by a mouthguard.

16. A data processing apparatus, comprising data processing resources configured to implement the method of claim 12 classifying impact data measured by an instrumented mouthguard.

17. A classifier comprising the data processing apparatus according to claim 16.
